(19) United States
(12) Patent Application Publication
(10) Pub. No.: US 2017/0015696 A1
(43) Pub. Date: Jan. 19, 2017

(54) SOLID STATE FORMS OF SOFOSBUVIR

(71) Applicants: RATIOPHARM GMBH, Ulm (DE); TEVA PHARMACEUTICALS INTERNATIONAL Gmbh, Jona (CH)

(72) Inventors: David Perez PALACIOS, Nen-ulm (DE); Jens GEIER, Oberdischingen (DE); Judith ARONHIME, Rehovot (IL); Limor TESSLER-SHAMIS, Netanya (IL); Sigalit LEVI, Modi'in (IL); Siva Rama Krishna MUPPALLA, Gajraula (IN)

(21) Appl. No.: 15/119,244

(22) PCT Filed: Feb. 19, 2015
(86) PCT No.: PCT/US2015/016473

§ 371 Date: Aug. 16, 2016

Related U.S. Application Data
(60) Provisional application No. 61/942,260, filed on Feb. 20, 2014.
(60) Provisional application No. 61/951,751, filed on Mar. 12, 2014.
(60) Provisional application No. 61/952,296, filed on Mar. 13, 2014.
(60) Provisional application No. 62/000,701, filed on May 20, 2014.
(60) Provisional application No. 62/008,184, filed on Jun. 5, 2014.

(51) Int. Cl. C07H 19/10

(52) U.S. Cl. CPC C07H 19/10; C07B 2200/13

(57) ABSTRACT

The present disclosure encompasses solid state forms of Sofosbuvir and pharmaceutical compositions thereof.

### CROSS REFERENCE TO RELATED APPLICATIONS

This application claims the benefit of U.S. Provisional Application Nos. 61/942,260, filed Feb. 20, 2014; 61/951,751, filed Mar. 12, 2014; 61/952,296, filed Mar. 13, 2014; 62/000,701, filed May 20, 2014; and 62/008,184, filed Jun. 5, 2014, the entireties of which are incorporated by reference herein.

### FIELD OF THE DISCLOSURE

The present disclosure encompasses solid state forms of Sofosbuvir and pharmaceutical compositions thereof.

### BACKGROUND OF THE DISCLOSURE

Sofosbuvir, L-Alanine, N-[[P(S),2′R]-2′-deoxy-2′-fluoro-2′-methyl-P-phenyl-5′-uridylyl]-, 1-methylethyl ester, or (2S)-isopropyl 2-(((((2R,3R,4R,5R)-5-(2,4-dioxo-3,4-dihydropyrimidin-1(2H)-yl)-4-fluoro-3-hydroxy-4-methyltetrahydrofuran-2-yl)methoxy)(phenoxy)phosphoryl)amino)propanoate, having the following formula,

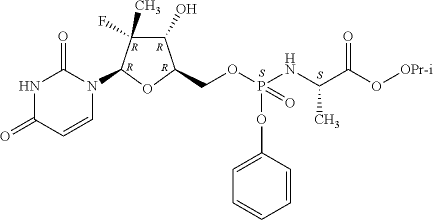

Sofosbuvir is an orally available, second generation uridine nucleoside analogue which inhibits the NS-5 protein of hepatitis C virus (HCV). Sofosbuvir and its isomer act as prodrugs and are converted through a series of in vivo transformations to an active triphosphate metabolite.

Sofosbuvir is described in U.S. Pat. No. 7,964,580 and in U.S. Pat. No. 8,334,270. Solid state forms of Sofosbuvir are described in WO 2010/135569, US 2011/251152, and WO 2011/123645.

Polymorphism, the occurrence of different crystalline forms, is a property of some molecules and molecular complexes. A single molecule may give rise to a variety of polymorphs having distinct crystal structures and physical properties like melting point, thermal behavior (e.g. measured by thermogravimetric analysis—“TGA”, or differential scanning calorimetry—“DSC”), X-ray diffraction pattern, infrared absorption fingerprint, and solid state (¹³C-) NMR spectrum. One or more of these techniques may be used to distinguish different polymorphic forms of a compound.

Different salts and solid state forms (including solvated forms) of an active pharmaceutical ingredient may possess different properties. Such variations in the properties of different salts and solid state forms and solvates may provide a basis for improving formulation, for example, by facilitating better processing or handling characteristics, changing the dissolution profile in a favorable direction, or improving stability (polymorph as well as chemical stability) and shelf-life. These variations in the properties of different salts and solid state forms may also offer improvements to the final dosage form, for instance, if they serve to improve bioavailability. Different salts and solid state forms and solvates of an active pharmaceutical ingredient may also give rise to a variety of polymorphs or crystalline forms, which may in turn provide additional opportunities to assess variations in the properties and characteristics of a solid active pharmaceutical ingredient.

Discovering new solid state forms and solvates of a pharmaceutical product may yield materials having desirable processing properties, such as ease of handling, ease of processing, storage stability, low hygroscopicity, and ease of purification or as desirable intermediate crystal forms that facilitate conversion to other polymorphic forms. New solid state forms of a pharmaceutically useful compound can also provide an opportunity to improve the performance characteristics of a pharmaceutical product. It enlarges the repertoire of materials that a formulation scientist has available for formulation optimization, for example by providing a product with different properties, e.g., a different crystal habit, higher crystallinity or polymorphic stability which may offer better processing or handling characteristics, improved dissolution profile, or improved shelf-life (chemical/physical stability). For at least these reasons, there is a need for additional solid state forms (including solvated forms) of Sofosbuvir.

### SUMMARY OF THE DISCLOSURE

The present disclosure provides solid state forms of Sofosbuvir, and pharmaceutical compositions and formulation comprising said solid state forms.

The present disclosure also encompasses the use of any one of the Sofosbuvir solid state forms of the present disclosure for the preparation of pharmaceutical compositions and pharmaceutical formulations of Sofosbuvir.

The present disclosure comprises a process for preparing the above mentioned pharmaceutical formulations. The process comprises combining any one of the Sofosbuvir solid state forms with at least one pharmaceutically acceptable excipient.

The solid state forms of Sofosbuvir and the pharmaceutical compositions and formulations comprising the solid state forms of Sofosbuvir of the present disclosure can be used as medicaments, particularly for the treatment of Hepatitis C.

The present disclosure also provides methods of treating Hepatitis C, comprising administering a therapeutically effective amount of any one of the crystalline forms of Sofosbuvir of the present disclosure, or at least one of the above pharmaceutical compositions or formulations, to a subject suffering from Hepatitis C, or otherwise in need of the treatment.

### BRIEF DESCRIPTION OF THE DRAWINGS

FIG. 1 depicts an X-ray powder diffractogram of form D of Sofosbuvir obtained by example 2.

FIG. 2 depicts an X-ray powder diffractogram of form C of Sofosbuvir obtained by example 3.

FIG. 3 depicts an X-ray powder diffractogram of form C of Sofosbuvir obtained by example 4.

FIG. 4 depicts an X-ray powder diffractogram of form 1 of Sofusbovir.

FIG. 5 depicts an X-ray powder diffractogram of amorphous Sofosbuvir.

FIG. 6 depicts an X-ray powder diffractogram of form C of Sofosbuvir obtained by example 3 in the range 2-30 deg two-theta.

FIG. 7 depicts an X-ray powder diffractogram of form D of Sofosbuvir obtained by example 2 in the range 2-30 deg two-theta.

FIG. 8 depicts a solid state ¹³C NMR spectrum of form D of Sofosbuvir obtained by example 2 in the range of 95-115 ppm

FIG. 9 depicts a solid state ¹³C NMR spectrum of form D of Sofosbuvir obtained by example 2 in the range of 10-30 ppm

FIG. 10 depicts an X-ray powder diffractogram of form D of Sofosbuvir obtained by example 10, in the range 2-30 deg two-theta.

FIG. 11 depicts an X-ray powder diffractogram of form A of Sofusbovir.

### DETAILED DESCRIPTION OF THE DISCLOSURE

The present disclosure encompasses solid state crystalline forms of Sofosbuvir. Solid state properties of Sofosbuvir can be influenced by controlling the conditions under which the Sofosbuvir is obtained in solid form.

In some embodiments, the crystalline forms of Sofosbuvir of the disclosure are substantially free of any other forms of Sofosbuvir, or of specified polymorphic forms of Sofosbuvir, respectively.

As used herein, “substantially free” is meant that the solid state forms of the present disclosure contain 20% (w/w) or less of polymorphs, or of a specified polymorph of Sofosbuvir. According to some embodiments, the salts and solid state forms of the present disclosure contain 10% (w/w) or less, 5% (w/w) or less, 2% (w/w) or less, 1% (w/w) or less, 0.5% (w/w) or less, or 0.2% (w/w) or less of polymorphs, or of a specified polymorph of Sofosbuvir. In other embodiments, solid state form of Sofosbuvir of the present disclosure contain from 1% to 20% (w/w), from 5% to 20% (w/w), or from 5% to 10% (w/w) of any solid state forms or of a specified polymorph of Sofosbuvir.

Depending on which other solid state forms comparison is made with, the crystalline forms of Sofosbuvir of the present disclosure have advantageous properties selected from at least one of the following: chemical purity, solubility, dissolution rate, morphology or crystal habit, stability—such as chemical stability as well as thermal and mechanical stability with respect to polymorphic conversion, stability towards solvation/desolvation and/or storage stability, low content of residual solvent, a lower degree of hygroscopicity, and advantageous processing and handling characteristics such as compressibility, flowability, and bulk density.

A solid state form, such as a crystal form or amorphous form, may be referred to herein as being characterized by graphical data “as depicted in” or “as substantially depicted in” a Figure. Such data include, for example, powder X-ray diffractograms and solid state NMR spectra. As is well-known in the art, the graphical data potentially provides additional technical information to further define the respective solid state form (a so-called “fingerprint”) which cannot necessarily be described by reference to numerical values or peak positions alone. In any event, the skilled person will understand that such graphical representations of data may be subject to small variations, e.g., in peak relative intensities and peak positions due to certain factors such as, but not limited to, variations in instrument response and variations in sample concentration and purity, which are well known to the skilled person. Nonetheless, the skilled person would readily be capable of comparing the graphical data in the Figures herein with graphical data generated for an unknown crystal form and confirm whether the two sets of graphical data are characterizing the same crystal form or two different crystal forms. A crystal form of a Sofosbuvir referred to herein as being characterized by graphical data “as depicted in” or “as substantially depicted in” a Figure will thus be understood to include any crystal forms of Sofosbuvir characterized with the graphical data having such small variations, as are well known to the skilled person, in comparison with the Figure.

As used herein, and unless stated otherwise, the term “anhydrous” in relation to crystalline forms of sofosbuvir, relates to a crystalline form of sofosbuvir which does not include any crystalline water (or other solvents) in a defined, stoichiometric amount within the crystal. Moreover, an “anhydrous” form would typically not contain more than 1% (w/w) of either water or organic solvents as measured for example by TGA.

As used herein, the term “isolated” in reference to solid state forms of Sofosbuvir of the present disclosure corresponds to a solid state form of Sofosbuvir that is physically separated from the reaction mixture in which it is formed.

As used herein, unless stated otherwise, the XRPD measurements are taken using copper Ka radiation wavelength of 1.5418 Å.

A thing, e.g., a reaction mixture, may be characterized herein as being at, or allowed to come to “room temperature” or “ambient temperature”, often abbreviated as “RT.” This means that the temperature of the thing is close to, or the same as, that of the space, e.g., the room or fume hood, in which the thing is located. Typically, room temperature is from about 20° C. to about 30° C., or about 22° C. to about 27° C., or about 25° C.

The amount of solvent employed in a chemical process, e.g., a reaction or a crystallization, may be referred to herein as a number of “volumes” or “vol” or “V.” For example, a material may be referred to as being suspended in 10 volumes (or 10 vol or 10V) of a solvent. In this context, this expression would be understood to mean milliliters of the solvent per gram of the material being suspended, such that suspending a 5 grams of a material in 10 volumes of a solvent means that the solvent is used in an amount of 10 milliliters of the solvent per gram of the material that is being suspended or, in this example, 50 mL of the solvent. In another context, the term “v/v” may be used to indicate the number of volumes of a solvent that are added to a liquid mixture based on the volume of that mixture. For example, adding solvent X (1.5 v/v) to a 100 ml reaction mixture would indicate that 150 mL of solvent X was added.

A process or step may be referred to herein as being carried out “overnight.” This refers to a time interval, e.g., for the process or step, that spans the time during the night, when that process or step may not be actively observed. This time interval is from about 8 to about 20 hours, or about 10-18 hours, typically about 16 hours.

As used herein, the term “reduced pressure” refers to a pressure that is less than atmospheric pressure. For example, reduced pressure is about 10 mbar to about 50 mbar.

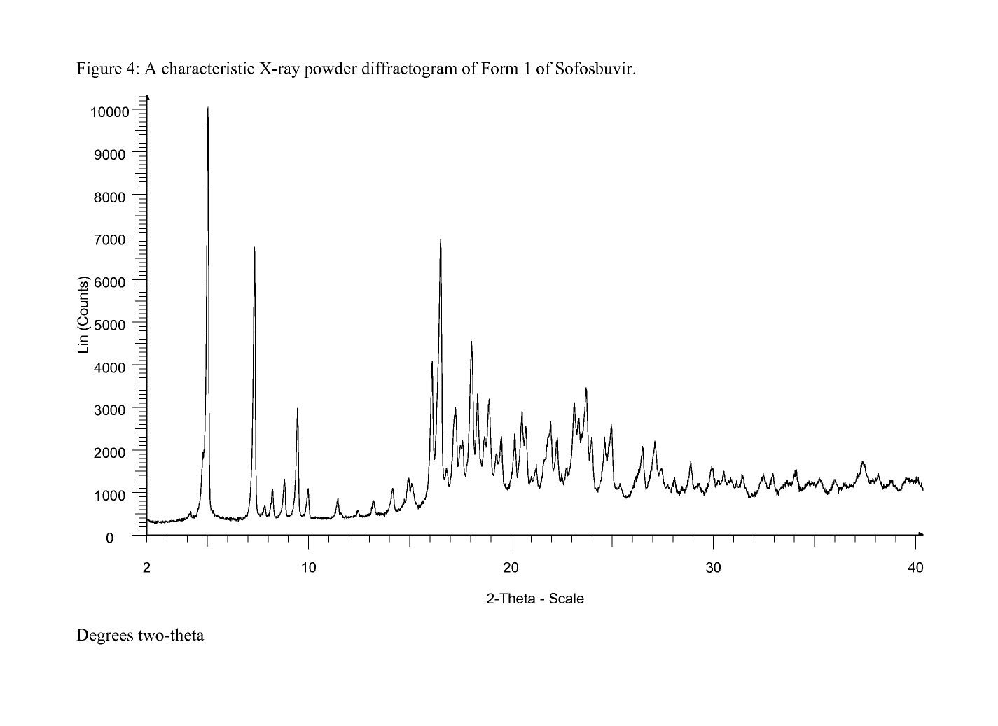

As used herein crystalline form 1 of Sofosbuvir refers to a crystalline form which may be characterized by X-ray powder diffraction pattern as depicted in FIG. 4.

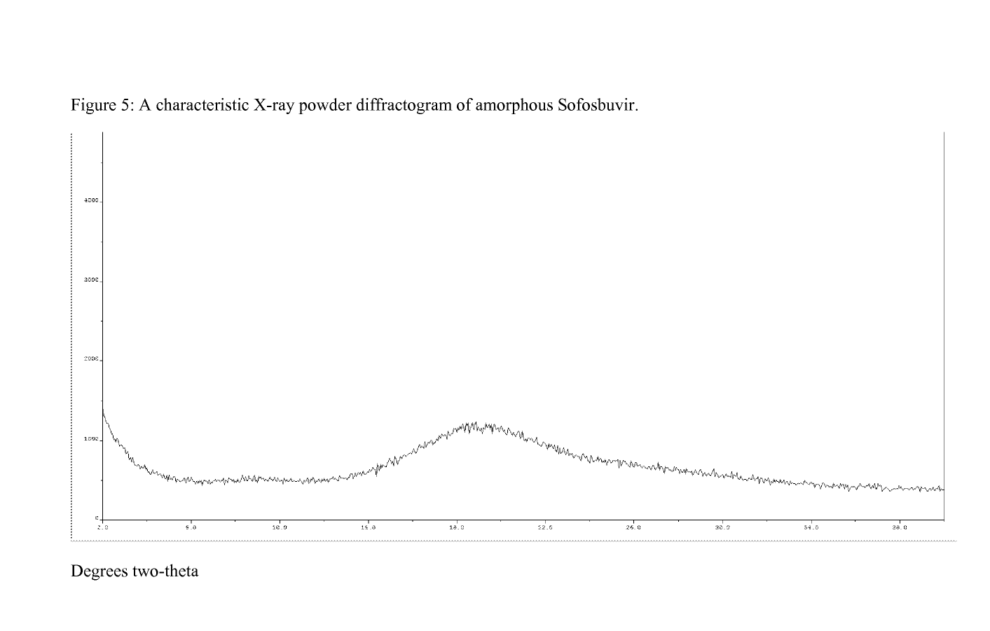

As used herein amorphous form of Sofosbuvir refers to an amorphous form which may be characterized by X-ray powder diffraction pattern as depicted in FIG. 5.

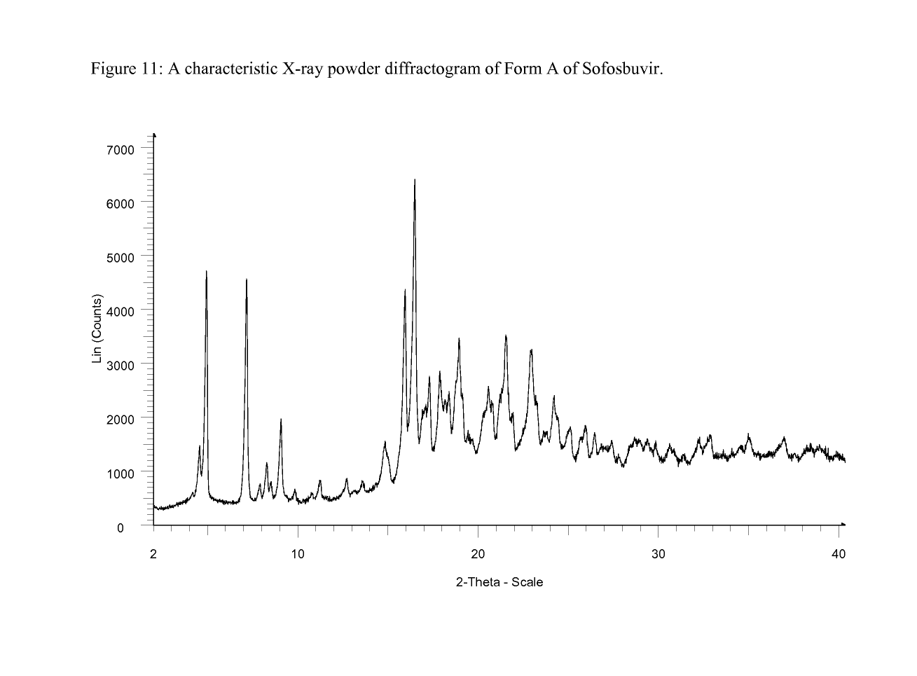

In embodiment first aspect, the present disclosure comprises a crystalline form of Sofusbovir, designated form A, characterized by data selected from one or more of the following: X-ray powder diffraction pattern having peaks at 4.5, 7.1, 9.0, 12.7 and 15.9 degrees two theta±0.2 degrees two theta; an X-ray powder diffraction pattern as depicted in FIG. 11; and combinations of these data.

Crystalline form A of Sofosbuvir may be further characterized by the X-ray powder diffraction pattern having peaks at 4.5, 7.1, 9.0, 12.7 and 15.9 degrees two theta±0.2 degrees two theta, and also having one, two, three, four or five additional peaks selected from: 4.9, 11.2, 13.6, 16.5 and 21.6±0.2 degrees two theta.

Crystalline form A of Sofosbuvir may be characterized by each of the above characteristics alone and/or by all possible combinations, e.g. by X-ray powder diffraction pattern having peaks at 4.5, 7.1, 9.0, 12.7 and 15.9 degrees two theta±0.2 degrees two theta and by an X-ray powder diffraction pattern as depicted in FIG. 11.

In one embodiment of the present disclosure, form A of Sofosbuvir is isolated.

Form A has certain advantageous properties as discussed above. Particularly, form A is stable for a period of 16 months when stored at ambient conditions (e.g., 1 atmosphere at RT).

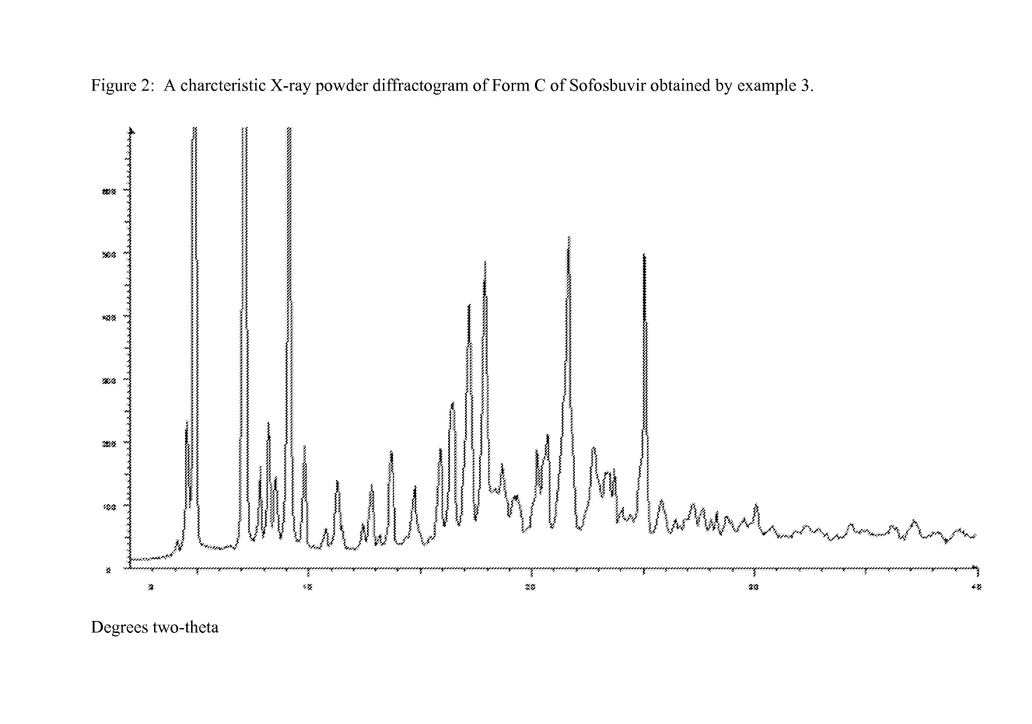

In another aspect, the present disclosure comprises a crystalline form of Sofusbovir, designated form C, characterized by an X-ray powder diffraction pattern as depicted in any one of FIG. 2, 3 or 6.

Crystalline form C of Sofosbuvir characterized by an X-ray powder diffraction pattern as depicted in any one of FIG. 2, 3 or 6, may be further characterized by X-ray powder diffraction pattern having one, two, three, four, five, six, seven, eight, nine or ten peaks selected from: 5.0, 7.2, 8.3, 9.3, 17.3, 20.3, 20.7, 21.8, 23.1 and 23.6 degrees two theta±0.1 degrees two theta.

Alternatively, crystalline form C of Sofosbuvir characterized by an X-ray powder diffraction pattern as depicted in any one of FIG. 2, 3 or 6, may be further characterized by X-ray powder diffraction pattern having one, two, three, four, five, six, seven, eight, nine or ten peaks selected from: 5.0, 7.2, 16.1, 17.3, 18.8, 19.0, 20.3, 20.7, 21.8 and 26.1 degrees two theta±0.1 degrees two theta.

Crystalline form C of Sofosbuvir may be characterized by each of the above characteristics alone and/or by all possible combinations.

In one embodiment of the present disclosure, form C of Sofosbuvir is isolated.

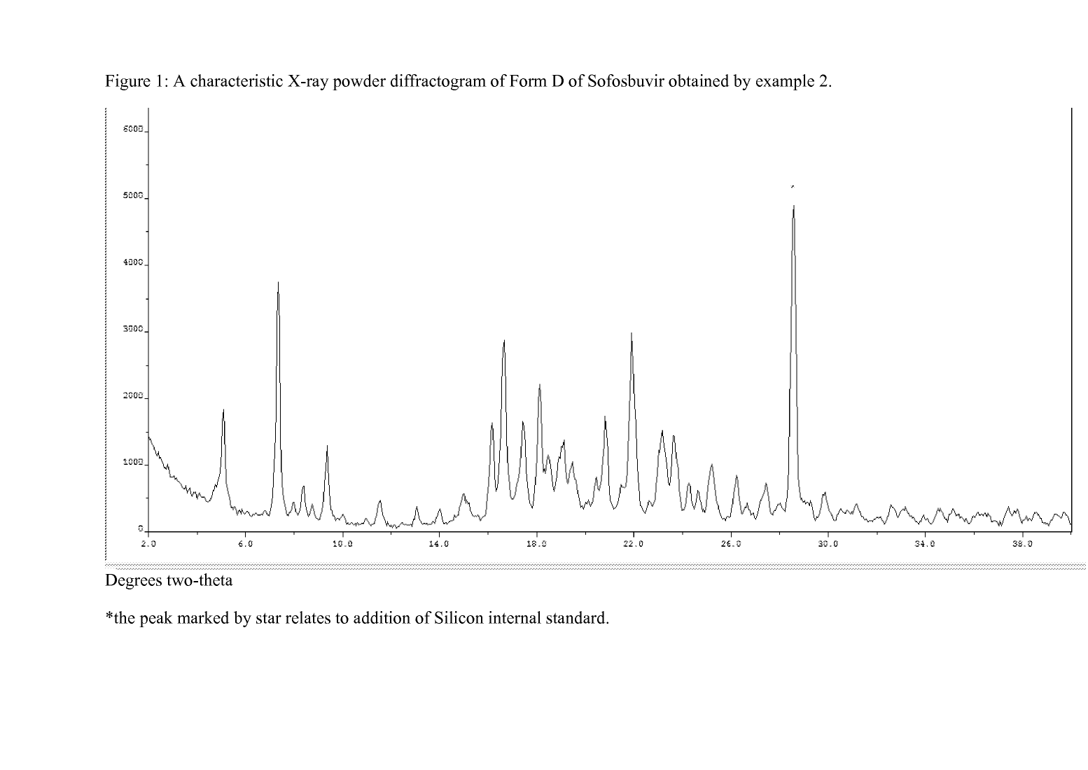

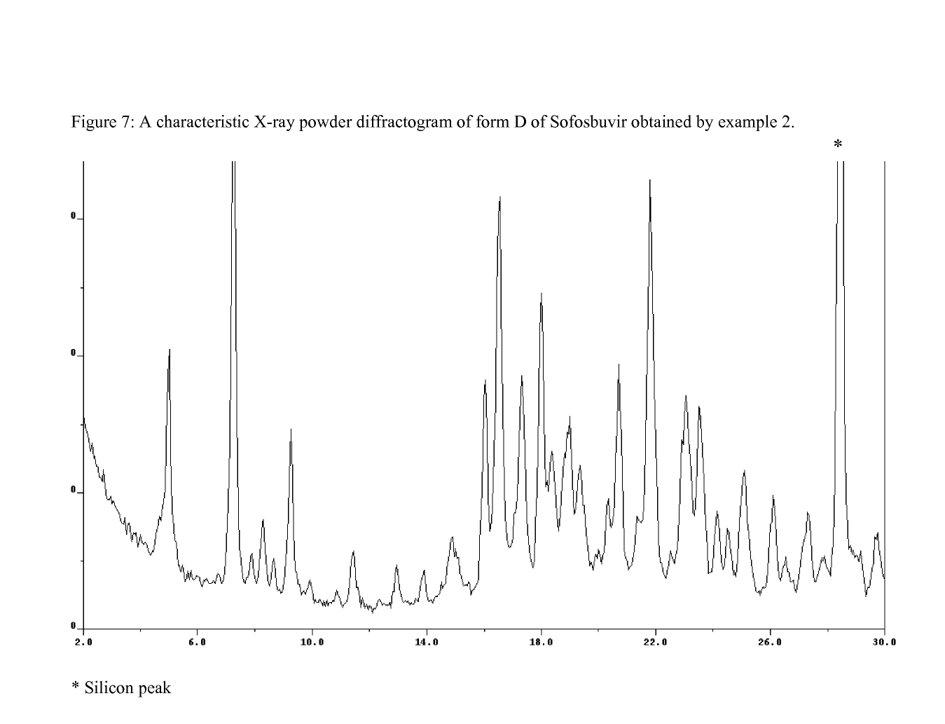

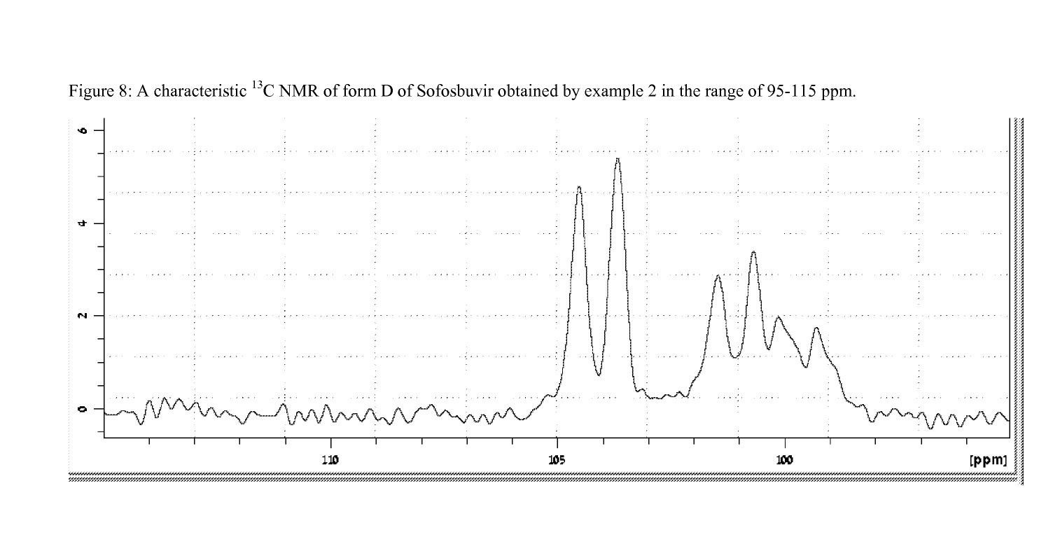

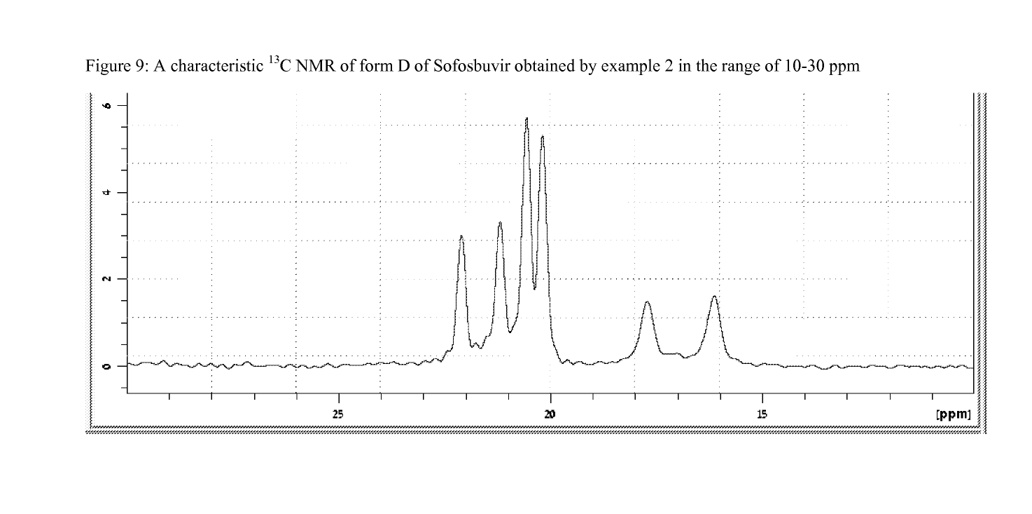

In another aspect, the present disclosure comprises crystalline form of Sofusbovir, designated form D, characterized by data selected from one or more of the following: an X-ray powder diffraction pattern as depicted in any one of FIG. 1 or 7; an X-ray powder diffraction pattern having characteristic peaks at: 16.0, 16.5, 17.3, 18.0, 18.4, 19.0, 20.7, 21.8, 23.0, 23.5 degrees two theta±0.1 degrees two theta and with an absence (not more than 5% relative intensity) of an XRPD peak at 17.6 degrees two theta±0.1 degrees two theta; a solid-state ¹³C NMR spectrum having two signals in the range 100-110 ppm at 104.4±0.3 ppm and 103.8±0.2 ppm; a solid-state ¹³C NMR spectrum having signals at 22.1 21.1, 20.5 and 20.2 ppm±0.2 ppm; a solid-state ¹³C NMR spectrum having chemical shifts differences between the signal at 16.1±0.2 and another peak of: 88.3±0.3 ppm and 87.7±0.2 ppm; a solid-state ¹³C NMR spectrum having chemical shifts differences between the signal at 16.1±0.2 ppm and another peak of: 6.0, 5.0, 4.4 and 4.1 ppm±0.2 ppm; a solid-state ¹³C NMR spectrum as depicted in any one of FIG. 8 or 9; and combinations of these data.

In some embodiments, crystalline form D of Sofosbuvir may be further characterized by an X-ray powder diffraction pattern having one, two, three, four, five, six, seven, eight, nine, ten, eleven or twelve peaks selected from: 13.0, 13.9, 16.0, 16.5, 17.3, 18.0, 18.4, 19.0, 20.7, 21.8, 23.0, 23.5 degrees two theta±0.1 degrees two theta.

Crystalline form D of Sofosbuvir may be characterized by each of the above characteristics alone and/or by all possible combinations.

In one embodiment of the present disclosure, form D of Sofosbuvir is isolated.

In another aspect, the present disclosure comprises pharmaceutical compositions and formulations comprising any one of the crystalline forms of Sofosbuvir of the present disclosure. Typically, the pharmaceutical composition is a solid composition and the Sofosbuvir retains its solid state form.

The pharmaceutical formulations can be prepared by a process comprising combining any one of the crystalline forms of Sofosbuvir of the present disclosure with at least one pharmaceutically acceptable excipient. The present disclosure further encompasses the use of the above-described crystalline forms of Sofosbuvir in the manufacture of a pharmaceutical composition or formulation as described herein.

Yet another aspect of the disclosure relates to the above crystalline forms of Sofosbuvir, or the pharmaceutical compositions or formulations comprising said crystalline forms of Sofosbuvir for use as a medicament. Preferably, said medicament can be used for the treatment of Hepatitis C.

The present disclosure further encompasses a method of treating a subject suffering from Hepatitis C, or otherwise in need of the treatment, comprising administration of an effective amount of the above crystalline forms of Sofosbuvir, or the pharmaceutical compositions or formulations comprising any one of the crystalline forms of Sofosbuvir described herein.

Having thus described the disclosure with reference to particular preferred embodiments and illustrative examples, those in the art can appreciate modifications to the disclosure as described and illustrated that do not depart from the spirit and scope of the disclosure as disclosed in the specification. The Examples are set forth to aid in understanding the disclosure but are not intended to, and should not be construed to limit its scope in any way.

### X-Ray Powder Diffraction

Samples were measured by an X-Ray diffractometer model X'TRA equipped with a solid state detector with Copper Kα radiation of 1.5418 Å. Scanning parameters: range: 2-40 degrees two-theta.

Silicon powder was added as internal standard for the measurement of forms C and D. The position of the silicon (111) peak was corrected to be 28.45 degrees two theta. The positions of the peaks of form C and D were corrected accordingly.

### Solid-State ¹³C NMR

Solid-state ¹³C NMR spectra were recorded with variable amplitude cross polarization, magic angle spinning and high power proton decoupling using a BRUKER Avance II+ spectrometer operating at 125 MHz and 0° C.—controlled temperature. A probe using 4 mm o.d. zirconia rotors was employed. The operation conditions were: contact time: 2 ms; recycle delay: 2 s; at least 1024 scans and spin rate of 11 kHz. Chemical shifts were referenced via a replacement sample of glycine (carboxyl carbon chemical shift assigned as 176.03 ppm relative to the signal of tetramethylsilane).

### EXAMPLES

### Reference Examples

Sofosbuvir form 1 was prepared according to the following procedure:

1-((2R,3R,4R,5R)-3-fluoro-4-hydroxy-5-(hydroxymethyl)-3-methyltetrahydrofuran-2-yl)pyrimidine-2,4(1H,3H)-dione (100 g) along with L-Alanine, N-[(S)-(2,3,4,5,6-pentafluorophenoxy) phenoxyphosphinyl]-, 1-methylethyl ester (209 g) was taken in a four necked round bottomed flask followed by addition of THF (1.5 L) at 25-30° C. under nitrogen atmosphere. Reaction mixture was subsequently cooled to −20° C. To the stirred solution was added dropwise 1.7 M t-butylmagnesium chloride (248 mL) in THF during 60-90 min maintaining the temperature of the reaction between −20 to −10° C. Reaction was then gradually warmed to 15-20° C. and stirred for another 5-20 h. After the completion of the reaction, 15% aq ammonium chloride solution (200 mL) was added dropwise at 0 to 10° C. to quench the reaction. THF was removed under vacuum and residue was dissolved in dichloromethane (1 L) and washed with 15% aq. ammonium chloride (1 L). Organic layer was then washed with 6% aq. sodium bicarbonate solution (2×1 L) and finally with 5% brine solution (1 L). Organic layer was filtered through celite and concentrated under vacuum. Dichloromethane/di-isopropyl ether (6:3, 900 mL) was added to the residue and the slurry was stirred for 10-12 h, filtered. The white solid was washed with 1:1 mixture of dichloromethane/di-isopropyl ether (400 mL). Wet cake thus obtained was again suspended in dicholoromethane (1 L). The slurry was stirred for 10-12 h at 20-25° C. Slurry was filtered and washed with dichloromethane (2×200 mL) to afford 125 g (61.4%) of sofosbuvir (Form-1).

Amorphous Sofosbuvir may be prepared according to the following procedure:

A 100 ml round bottom flask was charged with Sofosbuvir form 1 (5 g, 1 eq, 9.44 mmol) and acetonitrile (ACN, 50 ml, 10 V). The mixture was heated to reflux to obtain a clear solution. The solvent was then evaporated at room temperature to give Sofosbuvir as a white solid. The obtained solid was dried at the oven under reduced pressure over night at room temperature to provide amorphous form of Sofosbuvir.

### Example 1

### Preparation of Crystalline Form C of Sofosbuvir

Xylene was put in a glass vial (˜0.5 ml) and Sofosbuvir form 1 (˜300mg) was put in another glass vial. Both vials were kept open inside a glass beaker sealed with foil and parafilm at RT. The obtained material after 3 days of exposure to saturated atmosphere of xylene was analyzed by XRPD. Crystalline form C of Sofosbuvir was obtained as confirmed by XRPD.

### Example 2

### Preparation of Crystalline Form D of Sofosbuvir

Anisole (1 ml) was added to amorphous Sofosbuvir (about 88 mg). The mixture was stirred for 16 hours at 120° C. in a 1 ml closed vial; a clear solution was obtained. Additional 278 mg of amorphous Sofosbuvir was added to the solution and the obtained slurry was kept with stirring for 20 hours at 25° C. The material was extracted and dried for 3 days at 50° C., with vacuum, and was kept for 2 days at RT in a closed vial, to provide pure crystalline form D of Sofosbuvir (as confirmed by XRPD; FIGS. 1 and 7?).

### Example 3

### Preparation of Crystalline Form C of Sofosbuvir

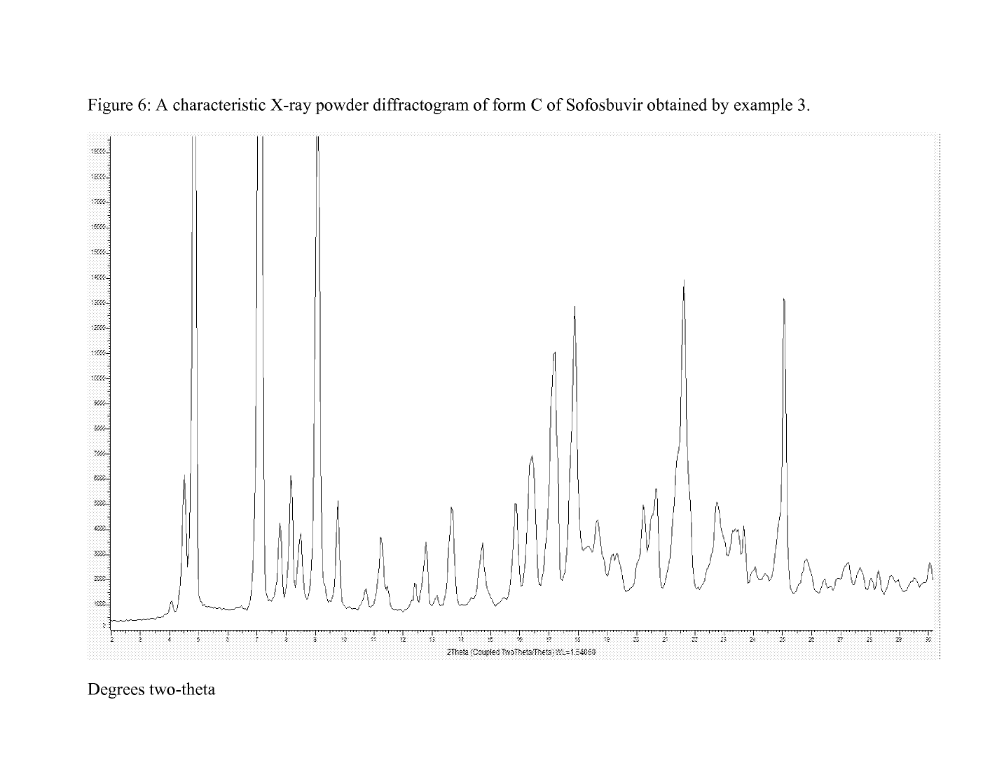

N-Heptane was put in a glass vial (˜2-3 ml) and Sofosbuvir form 1 (˜300mg) in another glass vial. Both vials were kept open inside a glass beaker sealed foil and parafilm at RT. The obtained material after 3 days of exposure to saturated atmosphere of N-heptane was analyzed by XRPD ; pure crystalline form C of Sofosbuvir was obtained (as confirmed by XRPD; FIGS. 2 and 6).

### Example 4

### Preparation of Crystalline Form C of Sofosbuvir

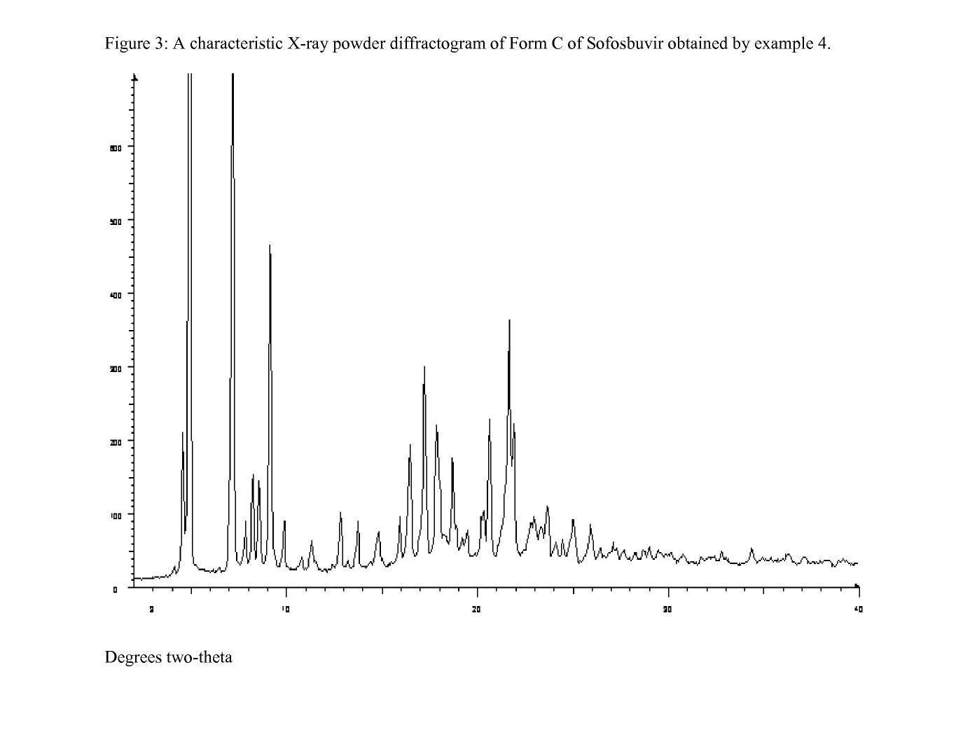

1-butanol was put in a glass vial (˜2-3 ml) and Sofosbuvir form 1 (˜300 mg) in another glass vial. Both vials were kept open inside a glass beaker sealed foil and parafilm at RT. The obtained material after 3 days of exposure to saturated atmosphere of 1-butanol was analyzed by XRPD; crystalline form C of Sofosbuvir was obtained (as confirmed by XRPD; FIG. 3).

### Example 5

### Preparation of Crystalline Form D of Sofosbuvir

MTBE (200 μL) was added to Sofosbuvir Form 1 (about 101 mg); a thick solution was obtained by stirring for one hour. After stirring for 1 hour, toluene (800 μL) was added to the solution. Colorless crystals were observed. The mixture was stirred for 10 days at 25° C. and for additional 2 days at 10° C. in a 1 ml closed vial. The material was filtered in centrifuge (1.5 min 2000 rpm) and dried over night at 60° C., form D was obtained (as confirmed by XRPD).

### Example 6

### Preparation of Crystalline Form D of Sofosbuvir

DIPE (300 μL) was added to Sofosbuvir Form 1 (about 100 mg); a thick solution was obtained by stirring for 1 hour. After stirring 1 hour, toluene (700 μL) was added to the solution. Colorless crystals were observed. The mixture was stirred for 10 days at 25° C. and then for additional 2 days at 10° C. in a 1 ml closed vial. The material was filtered in centrifuge (1.5 min 2000 rpm) and dried over night at 60° C., form D was obtained (as confirmed by XRPD).

### Example 7

### Preparation of Crystalline Form D of Sofosbuvir

MTBE (200 μL) was added to Sofosbuvir Form 1 (about 100 mg); a thick solution was obtained by stirring for 1 hour. After stirring 1 hour, n-Hexane (800 μL) was added to the solution. Colorless crystals were observed. The mixture was stirred for 10 days at 25° C. and for additional 2 days at 10° C. in a 1 ml closed. The material dried over night at 60° C., form D was obtained (as confirmed by XRPD).

### Example 8

### Preparation of Crystalline Form D of Sofosbuvir

An MTBE:Toluene mixture (0.5:0.5 ml) was added to Sofosbuvir form 1 (about 102 mg). The mixture was stirred for 20 hours at 25° C. in a 1 ml closed; a slurry was obtained. The supernatant solvent was removed with a pipette and the wet material was dried overnight at 50° C. in a vacuum oven. Form D was obtained (as confirmed by XRPD).

### Example 9

### Preparation of Crystalline Form D of Sofosbuvir

MTBE:n-Hexane mixture (0.5:0.5 ml) was added to Sofosbuvir form 1 (about 103 mg). The mixture was stirred for 20 hours at 25° C. in a 1 ml closed vial; a slurry was obtained. The supernatant solvent was removed with a pipette and the wet material was dried for overnight at 50° C. vacuum oven. Form D was obtained (as confirmed by XRPD).

### Example 10

### Preparation of Crystalline Form D of Sofosbuvir

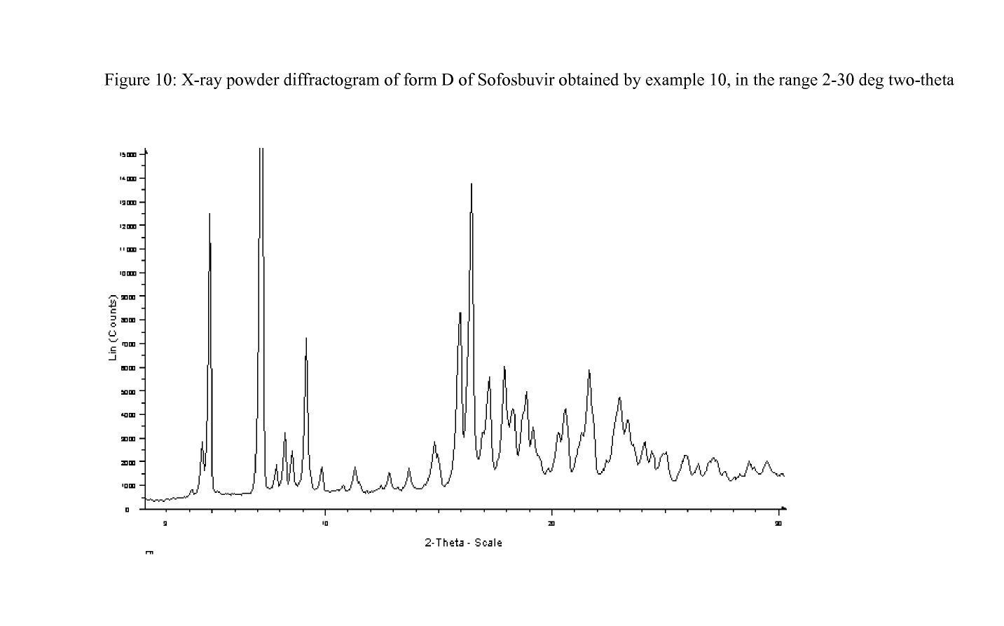

Sofosbuvir (Form-1, 1 g) was suspended in hexane (50 mL) and stirred at 25° C. for 72 h. The mass was filtered in a Buchner funnel and then suck dried for 15 min. The white solid obtained was analysed by XRPD to provide form D of Sofosbuvir (FIG. 10).

### Example 11

### Preparation of Crystalline Form A of Sofosbuvir

Form 1 of Sofosbuvir (400 mg) was suspended in diisopropyl ether (3 mL) and magnetically stirred for 24 hours at room temperature. The solid was isolated by filtration and dried at room temperature for 2 hours at 10 mbar. Form A was obtained as a colorless powder. The X-ray powder diffractogram of Form A is depicted in FIG. 11.

### Example 12

### Preparation of Crystalline Form A of Sofosbuvir

Form 1 of Sofosbuvir (200 mg) was suspended in cyclohexane (2 mL) and magnetically stirred for 72 hours at room temperature. The solid was isolated by filtration and dried at room temperature and atmospheric pressure for several hours. Form A was obtained as a colorless powder (as confirmed by XRPD).

### Example 13

### Preparation of Crystalline Form A of Sofosbuvir

Form 1 of Sofosbuvir (200 mg) was suspended in a 1:1 (v/v) mixture of tert.-butyl methyl ether and cyclohexane (2 mL) and magnetically stirred for 72 hours at room temperature. The solid was isolated by filtration and dried at room temperature and atmospheric pressure for several hours. Form A was obtained as a colorless powder (as confirmed by XRPD).

### Example 14

### Preparation of Crystalline Form A of Sofosbuvir

A filtered solution of Form 1 of Sofosbuvir (200 mg) in a 1:1 (v/v) mixture of tert-butyl methyl ether and n-heptane (40 mL) was stored in an open flask at room temperature and atmospheric pressure. After total evaporation of the solvent (3 days) Form A remained as a colorless solid (as confirmed by XRPD).

### Example 15

### Preparation of Crystalline Form A of Sofosbuvir

Two drops of DIPE or cyclohexane were added to Sofosbuvir form 1 (about 100 mg). The material was then ground using mortar and pestle for 1 minute to provide crystalline form A of Sofosbuvir (as confirmed by XRPD).

1. A crystalline form D of Sofosbuvir, characterized by data selected from the group consisting of:
an X-ray powder diffraction pattern as depicted in any one of FIG. 1 or 7;
an X-ray powder diffraction pattern having peaks at: 16.0, 16.5, 17.3, 18.0, 18.4, 19.0, 20.7, 21.8, 23.0, 23.5 degrees two theta±0.1 degrees two theta with not more than 5% relative intensity of an XRPD peak at 17.6 degrees two theta±0.1 degrees two theta;
a solid-state ¹³C NMR spectrum having two signals in the range 100-110 ppm at 104.4±0.3 ppm and 103.8±0.2 ppm;
a solid-state ¹³C NMR spectrum having signals at 22.1 21.1, 20.5 and 20.2 ppm±0.2 ppm;
a solid-state ¹³C NMR spectrum having chemical shifts differences between the signal at 16.1±0.2 and another peak of: 88.3±0.3 ppm and 87.7±0.2 ppm;
a solid-state ¹³C NMR spectrum having chemical shifts differences between the signal at 16.1±0.2 ppm and another peak of: 6.0, 5.0, 4.4 and 4.1 ppm±0.2 ppm;
a solid-state ¹³C NMR spectrum as depicted in any one of FIG. 8 or 9;
and combinations thereof.

2. The crystalline form according to claim 1, further characterized by an X-ray powder diffraction pattern having one, two, three, four, five, six, seven, eight, nine, ten, eleven, or twelve peaks selected from the group consisting of: 13.0, 13.9, 16.0, 16.5, 17.3, 18.0, 18.4, 19.0, 20.7, 21.8, 23.0, 23.5 degrees two theta±0.1 degrees two theta.

3. A crystalline form A of Sofosbuvir, characterized by data selected from the group consisting of:
an X-ray powder diffraction pattern having peaks at 4.5, 7.1, 9.0, 12.7 and 15.9 degrees two theta±0.2 degrees two theta;
an X-ray powder diffraction pattern as depicted in FIG. 11;
and combinations thereof.

4. The crystalline form according to claim 3, further characterized by an X-ray powder diffraction pattern having one, two, three, four or five additional peaks selected from the group consisting of: 4.9, 11.2, 13.6, 16.5 and 21.6±0.2 degrees two theta.

5. A pharmaceutical composition comprising the crystalline form of sofosbuvir according to claim 1.

6. A pharmaceutical formulation comprising the crystalline form of sofosbuvir according to claim 1, and at least one pharmaceutically acceptable excipient.

7. A process for preparing a pharmaceutical formulation, comprising combining one or more crystalline forms of sofosbuvir according to claim 1 and at least one pharmaceutically acceptable excipient.

8. (canceled)

9. (canceled)

10. (canceled)

11. A method of treating Hepatitis C in a subject comprising administering to the subject a therapeutically effective amount of the crystalline form of Sofosbuvir according to claim 1.

12. A pharmaceutical composition comprising the crystalline form of sofosbuvir according to claim 3.

13. A pharmaceutical formulation comprising the crystalline form of sofosbuvir according to claim 3 and at least one pharmaceutically acceptable excipient.

14. A pharmaceutical formulation comprising the pharmaceutical composition according to claim 5, and at least one pharmaceutically acceptable excipient.

15. A pharmaceutical formulation comprising the pharmaceutical composition according to claim 12, and at least one pharmaceutically acceptable excipient.

16. A process for preparing a pharmaceutical formulation, comprising combining one or more crystalline forms of sofosbuvir according to claim 3, and at least one pharmaceutically acceptable excipient.

17. A process for preparing a pharmaceutical formulation, comprising combining the pharmaceutical composition according to claim 5, and at least one pharmaceutically acceptable excipient.

18. A process for preparing a pharmaceutical formulation, comprising combining the pharmaceutical composition according to claim 12, and at least one pharmaceutically acceptable excipient.

19. A method of treating Hepatitis C in a subject comprising administering to the subject a therapeutically effective amount of the crystalline form of Sofosbuvir according to claim 3.

20. A method of treating Hepatitis C in a subject comprising administering to the subject a therapeutically effective amount of the pharmaceutical composition according to claim 5 or claim 12.

21. A method of treating Hepatitis C in a subject comprising administering to the subject a therapeutically effective amount of the pharmaceutical formulation according to claim 6, claim 13, claim 14, or claim 15.
